(19) United States
(12) Patent Application Publication
(10) Pub. No.: US 2022/0153803 A1
(43) Pub. Date: May 19, 2022

(54) MODIFIED GLUCAGON MOLECUES AND FORMULATIONS WITH OXIDATION RESISTANCE AND METHODS AND KITS OF EMPLOYING THE SAME

(71) Applicants: Purdue Research Foundation, West Lafayette, IN (US); Monon Bioventures, LLC, Fishers, IN (US)

(72) Inventors: Elizabeth M. Topp, Dublin (IE); Mark L. Heiman, Indianapolis, IN (US)

(21) Appl. No.: 17/439,732

(22) PCT Filed: Mar. 16, 2020
(86) PCT No.: PCT/US2020/022951

§ 371 Date: Sep. 15, 2021

Related U.S. Application Data
(60) Provisional application No. 62/818,826, filed on Mar. 15, 2019.

(51) Int. Cl. C07K 14/605; A61P 3/10

(52) U.S. Cl. CPC C07K 14/605; A61K 38/00; A61P 3/10

(57) ABSTRACT

Modified glucagon molecules and buffer and/or excipient solutions are provided that result in the glucagon molecules being resistant to oxidation when stored at a substantially neutral pH. Such a modified glucagon molecule includes a substitution at position 27, with the native methionine being replaced with a methionine memetic analog, a norleucine, or an isomer of either of the foregoing. Optionally, the modified glucagon molecules may be further phosphorylated to result in enhanced solubility at a substantially neutral pH and resistance to fibrillation. Methods of using such molecules in pharmaceutical compositions and therapeutic kits are also provided.

### PRIORITY

This application is related to and claims priority benefit of U.S. Provisional Patent Application Ser. No. 62/818,826 to Topp et al. filed Mar. 15, 2019. This application is further related, but does not claim priority, to U.S. patent application Ser. No. 15/745,483 to Topp et al., filed Jan. 17, 2018 and now patented as U.S. Pat. No. 10,308,701, which is a 371 national stage application, and claims the priority benefit of, International Patent Application No. PCT/US2016/043495 to Topp et al., filed Jul. 22, 2016, which is related to and claims the priority benefit of 62/195,537 to Topp et al, filed Jul. 22, 2015 (collectively, the “Related Disclosures”). The contents of the aforementioned applications are hereby incorporated by reference in their entireties into this disclosure.

### STATEMENT OF GOVERNMENT SUPPORT

This invention was made with government support under R44DK121594-01 awarded by the National Institute of Health Small Business Innovation Research. The government has certain rights in the invention.

### BACKGROUND

Diabetes is a chronic disease that occurs either when the pancreas does not produce enough insulin or when the body cannot effectively use the insulin it produces. Insulin is a hormone that regulates blood sugar. Type 1 diabetes is characterized by deficient insulin production and requires daily administration of insulin. The cause of type 1 diabetes is currently unknown and it is not preventable with current knowledge; however, the condition can be managed. Type 2 diabetes results from the body's ineffective use of insulin. Type 2 diabetes comprises the majority of people with diabetes around the world.

An estimated 30 million Americans have type 1 or type 2 diabetes, with 1.5 million newly diagnosed cases each year in the United States. These individuals must maintain a strict routine involving diet, exercise, medicines, and blood glucose monitoring to ensure that blood glucose is maintained at a healthy level. If this strict regimen is not followed, diabetics may experience severe to moderate hypoglycemia. Symptoms of mild to moderate hypoglycemia include headaches, blurred vision, dizziness, sweating, weakness, and confusion; in these cases, blood glucose levels can usually be restored with the ingestion of carbohydrates. However, in severe hypoglycemia (which occurs at least once a year for 40% of type 1 diabetics and for 20% of type 2 diabetics), symptoms are much more debilitating with individuals experiencing seizures and unconsciousness.

In severe hypoglycemia, an individual must be administered an intramuscular or subcutaneous injection of glucagon, a hormone that converts stored glycogen into glucose to be released into the bloodstream. While glucagon is effective at restoring blood sugar levels, a hypoglycemic event can result in a coma or death if administration is delayed or performed improperly. These cases then lead to a substantial economic impact: $120 million in emergency room visits and billions of dollars in hospitalizations are expended each year to treat severe hypoglycemic episodes. These costs are only expected to increase due to the growing population of individuals with diabetes.

Glucagon has long been used as a critical care medicine in the treatment of life-threatening hypoglycemia. Glucagon is a 29-residue peptide hormone secreted by pancreatic a-cells that plays an important role in glucose metabolism. It is commercially in a kit to be carried by the diabetic individual and typically provided as a lyophilized powder intended to be solubilized in dilute aqueous hydrochloric acid immediately prior to administration.

A significant problem with glucagon is that the molecule has poor water solubility at neutral pH and has to be solubilized in acidic pH. However, it is not stable even in an acidic solution, in which it irreversibly forms insoluble amyloid β-fibrils. Glucagon amyloid fibril formation compromises the potency of the drug, has the potential to generate toxic effects, and increases solution viscosity which causes difficulty in delivering the formulation using an infusion pump or injection pen.

Accordingly, due to these solubility and stability issues, the commercially available kits consist of glucagon formulated as a lyophilized powder and a syringe prefilled with solvent, such that the glucagon can be reconstituted just prior to administration and any surplus solution discarded immediately thereafter.

When an individual experiences severe hypoglycemia, it is a high stress, emergency situation. The conventional approach using emergency kits thus requires a third party to navigate a several step procedure under highly stressful conditions to successfully reconstitute the glucagon and administer the dose. The anxiety of the third party can lead to non-use of the kit or errors in administration, with errors occurring in upwards of 30% of kit uses. The inconvenience and risk of needle exposure and dosing error associated with conventional formulations has led to underutilization of glucagon despite its safety and efficacy for treatment of hypoglycemia. Additionally, kits are often not available when needed, since individuals feel they are cumbersome to carry due to their size, which leads to increased reliance upon emergency rooms for hypoglycemic rescue. The under-utilization of conventional kits and the rate of errors in such kit use highlight the need for improvements in glucagon rescue strategies. Indeed, to improve outcomes for individuals experiencing severe hypoglycemic events and reduce overall healthcare expenditures, what is needed is a simple, easy to use, and cost-effective solution that promotes more wide-spread and effective usage.

Furthermore, glucagon solubility and stability issues have hindered the development of a closed loop artificial pancreas device. Such a device could administer insulin and glucagon automatically in response to fluctuations in blood glucose and could significantly improve quality of life for diabetic patients. It is impractical to use the lyophilized glucagon formulation for an artificial pancreas, which requires that an adjustable amount of glucagon solution be administered instantaneously in response to fluctuations in blood glucose. Accordingly, a stable and safe glucagon alternative is needed to realize the potential benefits of an artificial pancreas device in treating diabetic patients.

### BRIEF SUMMARY

The present disclosure provides modified glucagon molecules and formulations that are soluble in an aqueous solution at a substantially neutral pH and are oxidation resistant.

Conventional solubility and stability issues for glucagon occur in part because glucagon fibrillates form amyloid β-fibrils. Amyloid β-fibrils are long β-sheets known as β-spines that interact side-by-side by entanglement of their side chains, forming a steric zipper. Aspects of the present disclosure are based on modifying certain amino acid residues of a glucagon molecule that interact with each other to form the steric zipper. Modification of those amino acids in a manner that prevents their interaction inhibits fibril formation and, thus promotes solubility of the molecule. Furthermore, in certain embodiments, to promote oxidative resistance, native glucagon or phosphoglucagon is stored in an antioxidant formulation, or the glucagon and/or phosphoglucagon is modified to replace the methionine residue. Formulating glucagon as a stable solution not only promotes its utilization for current uses, but also is a major step toward expanding glucagon's therapeutic benefits through artificial pancreas devices and otherwise.

In at least one exemplary embodiment of the present disclosure, a peptide is provided comprising SEQ ID NO: 1 (native glucagon) modified such that the molecule is soluble at a substantially neutral pH and/or resistant to oxidation (over time). An exemplary modification is one in which the one or more amino acids have been reversibly phosphorylated to prevent the formation of amyloid fibrils and further the methionine at position 27 thereof has been substituted to reduce oxidation over time. Position 27 may be substituted with an oxidation resistant methionine memetic analog or an isomer thereof. In at least one embodiment, the methionine memetic analog comprises norleucine or an isomer thereof, or methoxinine or an isomer thereof. In at least one exemplary embodiment, the peptide comprises SEQ ID NO: 2, wherein X comprises norleucine or an isomer thereof, or methoxinine or an isomer thereof.

Where the peptide is phosphorylated at one or more amino acids, such amino acids are selected from the group consisting of His¹, Ser², Thr⁵, Thr⁷, Ser⁸, Tyr¹⁰, Ser¹¹, Tyr¹³, Ser¹⁶, Thr²⁹, and combinations thereof.

Pharmaceutical compositions are also provided. In at least one embodiment, a pharmaceutical composition of the present disclosure comprises a modified peptide or pharmaceutically acceptable salt thereof, the modified peptide comprising SEQ ID NO: 1 modified such that (a) the amino acid at position 27 is substituted with an oxidation resistant methionine memetic analog or an isomer thereof, (b) one or more of the amino acids of the modified peptide are phosphorylated (e.g., and without limitation, at the amino acid residues listed above), or (c) both (a) and (b); and a pharmaceutically acceptable carrier.

In certain embodiments, such pharmaceutical composition may further comprise an antioxidant. Such antioxidant may comprise ascorbic acid, cysteine, polysorbate 20, polysorbate 80, ethylenediaminetetraacetic acid (EDTA), methionine, and/or an isomer of any of the foregoing antioxidants. In at least one exemplary embodiment, the pharmaceutical composition comprises phosphate-buffered saline (PBS) with 1-5 mM EDTA suspended therein, PBS with 0.5 mM-50 mM L-methionine suspended therein, histidine buffer with 1-5 mM EDTA suspended therein, or histidine buffer with 0.5 mM-50 mM L-methionine suspended therein.

In still other embodiments, the composition may comprise a prodrug. For example, and without limitation, in the prodrug, each phosphate group is chemically or enzymatically cleaved upon administration of the prodrug.

The pharmaceutical composition may comprise an aqueous solution at a substantially neutral pH. Additionally or alternatively, the pharmaceutical composition may comprise the modified peptide in a concentration of at or between 1 mg/mL-50 mg/mL.

Methods of treating a condition using the modified peptides and formulations of the present disclosure are also provided. In at least one embodiment, the method comprises treating a condition or a complication thereof by administering to a subject a stable formulation comprising a modified glucagon in an amount effective to treat the condition (e.g., gastrointestinal motility or a diabetic condition). There, the glucagon is modified such that (a) an amino acid at position 27 is substituted with an oxidation resistant methionine memetic analog or an isomer thereof, (b) one or more amino acids of the glucagon are phosphorylated, (c) or both (a) and (b). The modified glucagon may comprise SEQ ID NO: 2, wherein X is norleucine or an isomer thereof or methoxinine or an isomer thereof. Additionally or alternatively, the stable formulation further comprises one or more antioxidants selected from the group consisting of: ascorbic acid, cysteine, polysorbate 20, polysorbate 80, EDTA, methionine, or an isomer of any of the foregoing, using any of the specific formulations referenced herein.

Additional methods provide for administering insulin to the subject. In at least one embodiment, the stable formulation of modified glucagon and insulin are administered at different times via a device that monitors blood glucose levels of the subject and doses the two drugs independently as needed.

Kits for treating a condition are also provided, such comprising a stable formulation of the present disclosure. In at least one exemplary embodiment, the stable formulation is an aqueous solution at a substantially neutral pH. Such kits may further comprise a vial, a cartridge, an auto-injector device, a pump, or a nasal spray device, all of which may store the stable formulation (i.e. premixed/prefilled). For example, and without limitation, the kit may comprise a syringe, wherein the syringe is prefilled with the stable formulation and the stable formulation further comprises an antioxidant. Further, the stable formulation may comprise a therapeutically effective dose of the modified peptide.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1, subpart A shows an example of an energetically favorable structure for a native glucagon fibril steric zipper region with a highly hydrophobic core, while subpart B shows a model of glucagon molecule with phosphate esters on Ser⁸ (a residue buried within the hydrophobic core), which places a charged group in the middle of the hydrophobic core thus preventing steric zipper formation;

FIG. 2 illustrates the amino acid sequence of native glucagon (SEQ ID NO: 1), with the ten amino acids identified as readily phosphorylatable side chains shown underlined;

FIG. 3 shows a graphical representation of the relative percent Met²⁷ oxidation in 1-month stability samples of a phosphor-Ser⁸-glucagon analog, with Met²⁷ oxidation quantified by measuring the peak height of oxidized species relative to non-oxidized species in the mass spectra;

FIG. 4 shows a graphical representation of blood glucose measurements in rats in response to administration of either native glucagon or phosphoglucagon (n=8 for each test group);

FIGS. 5A-5I show CD spectra results from weeks 0 to 12, with FIG. 5A representative of a phospho-Thr⁵-glucagon, FIG. 5B showing phospho-Thr⁵-glucagon in an ethylenediaminetetraacetic acid (EDTA) solution, FIG. 5C showing phospho-Thr⁷-glucagon, FIG. 5D showing phospho-Thr⁷-glucagon in an EDTA solution, FIG. 5E showing phospho-Ser⁸-glucagon, FIG. 5F showing phospho-Ser⁸-glucagon in an EDTA solution; FIG. 5G showing phospho-Thr⁵-glucagon with Met²⁷ substituted for Nle²⁷; FIG. 5H showing phospho-Thr⁷-glucagon with Met²⁷ substituted for Nle²⁷, FIG. 5I showing phospho-Ser⁸-glucagon with Met²⁷ substituted for Nle²⁷;

FIGS. 6A and 6B illustrate mass spectrometry results of the samples of FIGS. 5A-5I, which support that either no or minimal oxidation or degradation occurred in the methionine substituted and antioxidant test samples by week 12.

While the present disclosure is susceptible to various modifications and alternative forms, exemplary embodiments thereof are shown by way of example in the drawings and are herein described in detail.

### BRIEF DESCRIPTION OF THE SEQUENCE LISTINGS

SEQ ID NO: 1 is an amino acid sequence of native glucagon: HSQGTFTSDYSKYLDSRRAQDFVQWLMNT; and

SEQ ID NO: 2 is an artificial amino acid sequence of methionine substituted glucagon, where X is a memetic analog of methionine, including and without limitation norleucine or an isomer thereof, or methoxinine or an isomer thereof:

HSQGTFTSDYSKYLDSRRAQDFVQWLXNT.

In addition to the foregoing, the above-described sequences are provided in computer readable form encoded in a file filed herewith and herein incorporated by reference. The information recorded in computer readable form is identical to the written Sequence Listings provided above, pursuant to 37 C.F.R. § 1.821(f).

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, systems and methods hereof may comprise many different configurations, forms, materials, and accessories.

In the following description, numerous specific details are set forth in order to provide a thorough understanding of the present disclosure. Particular examples may be implemented without some or all of these specific details and it is to be understood that this disclosure is not limited to particular biological systems, which can, of course, vary.

Unless defined otherwise, all technical and scientific terms used herein have the same meaning as commonly understood by one of skill in the relevant arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

A “subject” or “patient” as the terms are used herein is a mammal. While preferably a human, the terms can also refer to a non-human mammal, such as a mouse, cat, dog, monkey, horse, cattle, goat, or sheep, and is inclusive of male, female, adults, and children.

As used herein, the phrase “diabetic condition” includes, without limitation, type 1 diabetes, type 2 diabetes, gestational diabetes, pre-diabetes, hypoglycemia, and metabolic syndrome.

The terms “treatment” or “therapy,” as used herein include curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. Prophylactic treatment refers to any of the following: halting the onset, reducing the risk of development, reducing the incidence, delaying the onset, reducing the development, and increasing the time to onset of symptoms of a particular disorder.

As used herein, the phrases “therapeutically effective dose,” “therapeutically effective amount,” and “effective amount” means (unless specifically stated otherwise) a quantity of a compound which, when administered either one time or over the course of a treatment cycle, affects the health, wellbeing or mortality of a subject (e.g., and without limitation, a diminishment or prevention of effects associated with a diabetic condition). The a appropriate dosage or amount of a peptide drug or other compound to be administered to a subject for treating a disease, condition, or disorder (including, without limitation, a diabetic condition) as described herein will vary according to several factors including the type and severity of condition being treated, how advanced the disease pathology is, the formulation of the composition, patient response, the judgment of the prescribing physician or healthcare provider, and the characteristics of the patient or subject being treated (such as general health, age, sex, body weight, and tolerance to drugs). A therapeutically effective amount is also one in which any toxic or detrimental effects of the therapeutic agent are outweighed by the therapeutically beneficial effects.

Further, administered dosages for the peptide drugs as described herein for treating a diabetic condition or other disease or disorder are in accordance with dosages and scheduling regimens practiced by those of skill in the art. General guidance for appropriate dosages of all pharmacological agents used in the present methods is provided in Goodman and Gilman's The Pharmacological Basis of Therapeutics, 11th Edition, 2006, supra, and in Physicians' Desk Reference (PDR), for example, in the 71st (2017) Ed. or those since made available online (PDR.net), PDR Network, LLC, each of which is hereby incorporated herein by reference.

Determining an effective amount or dose is well within the capability of those skilled in the art, especially in light of the detailed disclosure provided herein. Generally, the formulations to deliver these doses may contain one, two, three, four, or more peptides or peptide analogs (collectively “peptide,” unless peptide analogs are expressly excluded), wherein each peptide is present at a concentration from about 0.1 mg/mL up to the solubility limit of the peptide in the formulation. This concentration is preferably from about 1 mg/mL to about 100 mg/mL, e.g., about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, and dispensing agents (depending on the nature of the mode of administration and dosage forms.

The term “pharmaceutically acceptable” and grammatical variations thereof, as they refer to compositions, carriers, diluents, reagents, and the like, are used interchangeably and represent that the materials are capable of administration to or upon a mammal without undue toxicity, irritation, allergic response, and/or the production of undesirable physiological effects such as nausea, dizziness, gastric upset, and the like as is commensurate with a reasonable benefit/risk ratio.

The term “phosphoglucagon” as used herein refers to a glucagon molecule derivative that has been phosphorylated at one or more amino acid side chains thereof as described in the Related Disclosures and herein.

The term “prodrug” as used herein refers to compounds that are rapidly transformed in vivo to yield the parent compound (here, native glucagon), for example by hydrolysis in blood. Functional groups that may be rapidly transformed in vivo by hydrolysis, metabolic cleavage, or other reactions can be used as derivatizing agents for prodrugs (i.e. “promoieties”). Promieties include, without limitation, such groups as alkanoyl (such as acetyl, propionyl, butyryl, and the like), unsubstituted and substituted aroyl (such as benzoyl and substituted benzoyl), alkooxycarbonyl (such as ethoxycarbonyl), trialkylsilyl (such as trimethyl- and triethysilyl), monoesters formed with dicarboxylic acids (such as succinyl), phosphate esters, sulfate esters and the like. Because of the ease with which the metabolically cleavable groups of the compounds useful according to the present disclosure are cleaved in vivo, the compounds bearing such groups act as prodrugs. The compounds bearing the metabolically cleavable groups have the advantage that they may exhibit improved bioavailability or other desirable properties as a result of enhanced solubility and/or rate of absorption conferred upon the parent compound by virtue of the presence of the metabolically cleavable group. A “true prodrug” is pharmacologically inactive in its derivatized form, gaining its activity only when the promoiety has been removed. However, as the term is used herein, “prodrug” refers to compound derivatized with promoieties that can be cleaved chemically or enzymatically in vivo, regardless of whether such compounds show activity in their derivatized forms. Thus, the term “prodrug” encompasses both “true prodrugs” and derivatives with cleavable promoieties that show activity in their derivatized form.

A “neutral pH” as used herein refers to a pH of about 7. A “substantially neutral pH” is a pH that may not be exactly a pH of 7, but also include a pH ranging between 4 and 9 and includes any value therebetween. A substantially neutral pH includes a physiological neutral pH of about 7.4.

As used herein, the phrase “chemical stability” means that, with respect to the therapeutic agent, an acceptable percentage of degradation products produced by chemical pathways such as oxidation or hydrolysis is formed when the formulation is stored under specific conditions. In some embodiments, a chemically stable formulation has less than 20%, less than 15%, less than 10%, less than 5%, less than 4%, less than 3%, less than 2%, or less than 1% breakdown products formed after an extended period of storage at the intended storage conditions of the product.

As used herein, the term “physical stability” means that, with respect to the therapeutic agent, an acceptable percentage of aggregates (e.g., dimers, trimers and larger forms) and other physical degradants (e.g., precipitate) is formed. In some embodiments, a physically stable formulation has less than 15%, less than 10%, less than 5%, less than 4%, less than 3%, less than 2%, or less than 1% aggregates or other physical degradation products formed after an extended period of storage at the intended storage conditions of the product.

As used herein, the term “stable formulation” means that the formulation maintains the chemical and physical stability of the active pharmaceutical ingredient (e.g., phosphoglucagon and/or a methionine substituted glucagon) to within acceptable limits after an extended period of storage at the intended storage conditions of the product. In some embodiments, a stable formulation has less than 10% degradation over two years or less than 5% degradation over two years.

The term “isolated” means that the material is removed from its original environment, e.g., the natural environment if it is naturally occurring. For example, a naturally-occurring polypeptide present within a living organism is not isolated, but the same polypeptide separated from some or all of the coexisting materials in the natural system is isolated.

The term “purified” does not require absolute purity; instead, it is intended as a relative definition.

The inventive concepts of the present disclosure generally relate to methods, compositions, and modified peptides that enhance the stability of solubilized glucagon as compared to native glucagon and previously described phosphoglucagon derivatives stored using conventional techniques. These inventive strategies minimize oxidation of glucagon to achieve such enhanced stability. Such methods, compositions, and modified peptides may be utilized with native glucagon as the starting point or, in an exemplary embodiment, applied in conjunction with the phosphoglucagon techniques described in the Related Disclosures to achieve not only enhanced stability, but also enhanced solubility at a neutral pH.

Native glucagon (SEQ ID NO: 1) is found to be soluble at a pH of 3 or below and at a pH of 10 and above. Without being bound by any particular theory or mechanism of action, it is believed that the solubility and stability issues associated with native glucagon at a substantially neutral pH are due to its near-neutral isoelectric point (PI) and to glucagon fibrillating and forming amyloid β-fibrils. Amyloid β-fibrils are long β-sheets known as β-spines that interact side-by-side by entanglement of their side chains forming a “steric zipper.”

As set forth in detail in the Related Disclosures, it has been determined that disrupting the steric zippers formed by native glucagon through the addition of phosphate to certain amino acid side chains improves the solubility and stability of the modified glucagon molecules as compared to native glucagon. At neutral pH, phosphorylation introduces negative charge (−2) into zipper-forming side chains, inhibiting glucagon self-association and fibrillation through charge repulsion and, thus, increasing solubility at neutral pH.

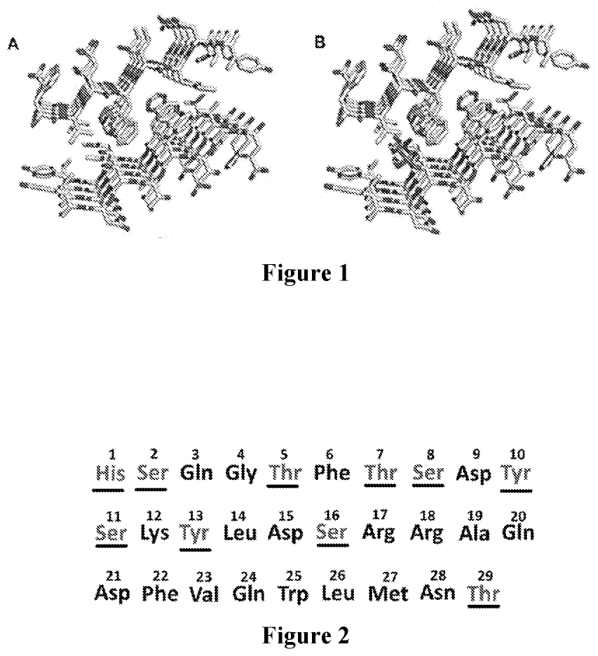

FIG. 1, subparts A and B illustrate how residues buried in the hydrophobic core of a glucagon molecule can be phosphorylated (Ser⁸ in this example). Phosphorylation places a charged group in the middle of the hydrophobic core, thereby preventing steric zipper formation. Computational models suggest that phosphorylation on Thr⁵ or Ser⁸ is more effective than on Ser² since those sites place the charge in the middle of the steric zipper as opposed to its side. Accordingly, phosphorylation of certain amino acid residues resulted in a modified glucagon that was soluble and stable at a substantially neutral pH (i.e. a pH between about 4-9).

Further, it was also determined that the phosphate group is easily removed enzymatically in phosphatase enzyme concentrations close to serum conditions, resulting in free native glucagon. In other words, upon injection, the phosphate moiety is cleaved by phosphatase enzymes naturally present throughout the body, thus regenerating native glucagon and promoting the conversion of glycogen to glucose to restore blood sugar levels.

This is essentially a pro-drug approach, long used to improve the solubility of small molecule drugs (for example, fosphenytoin), and here applied to inhibiting glucagon fibrillation. As alluded to above, phosphorylation also increases the solubility of glucagon at a neutral pH by shifting its isoelectric point (PI). The theoretical PI of glucagon is near 7 so that the molecule is essentially uncharged and least soluble in neutral aqueous solutions. Adding a single phosphate group decreases the net charge by 2, increasing the solubility at neutral pH.

The phosphorylation process is well known in the art and can be accomplished using known techniques. In one embodiment, phosphorylation of the targeted amino acids can be accomplished as a reversible enzymatic process that involves kinase and phosphatase enzymes in a process in which ATP acts as a phosphoryl donor. The overall reaction can be represented as follows:

Phosphorylation: E+ATP→→E-P+ADP

Further, phosphoglucagons may be prepared by solid-phase or other well-known peptide synthesis procedures using one or more phosphorylated amino acids as reagents.

Without limiting derivatization to these amino acids, it is noted that there are 10 readily phosphorylatable amino acid side chains on native glucagon (i.e. His¹, Ser², Thr⁵, Thr⁷, Ser⁸, Tyr¹⁰, Ser¹¹, Tyr¹³, Ser¹⁶, Thr²⁹) (see FIG. 2). Of the 10 residues, two are at the chain termini (His¹ and Thr²⁹) and less likely to be involved in fibril formation. Nevertheless, there are 10 singly phosphorylated, 45 doubly phosphorylated, and 120 triply phosphorylated possible glucagon prodrugs carrying between one and three phosphate groups on these readily phosphorylatable sites, which is a total of 175 distinct molecules. Allowing for up to ten sites of phosphorylation, the number of distinct phosphoglucagon derivatives based on the readily phosphorylatable side chains increases to 1,023. These phosphoglucagon derivatives have proven to have enhanced solubility and stability over their native glucagon counterparts. Furthermore, these compounds are effective in vivo. In fact, the data presented herein and the Related Disclosures show that the inventive phosphoglucagons are readily dephosphorylated following administration to a subject such that they revert to native glucagon. In addition, their performance in vivo is comparable to native glucagon.

Native glucagon (SEQ ID NO: 1) and, thus, the phosphoglucagons previously described, include a methionine residue at position 27, which is an amino acid that is prone to oxidation by reactive oxygen species (ROS). Oxidation can lead to protein misfolding, which can negatively affect the stability of glucagon and/or impair its biological function and have a significant influence over its immunogenicity. Despite the favorable preliminary research relating to phosphoglucagon, the phosphoglucagon analogs have shown oxidation of Met²⁷ to methionine sulfoxide and (to a lesser extent) methionine sulfone after 30 days of storage in unprotected formulations. To extend the shelf-life of the formulation and make phosphoglucagons and/or native glucagon amendable to other applications (such as for use in an artificial pancreas), the stability of the current formulation must be further extended. As presented herein, this can be achieved through modifications of the methionine residue of either phosphorylated or native glucagon and/or through the use of novel antioxidant-rich formulations.

To address stability concerns, in at least one exemplary embodiment of the present disclosure, a glucagon molecule is provided that includes an amino acid substitution at position 27 (methionine or Met²⁷) to enhance the chemical stability of the molecule (SEQ ID NO: 2). For example, Met²⁷ may be substituted with an oxidation-stable methionine memetic analog or an isomer thereof.

In certain embodiments of the present disclosure, norleucine (Nle²⁷), methoxinine (Mox²⁷) (also called homoserine methyl ether), or isomers of Nle or Mox may be substituted for the Met²⁷. Norleucine is similar to methionine in several respects, however, due to having a different side chain it is less susceptible to oxidation. A Met→Nle switch preserves the length of the amino acid side chain that is important for hydrophobic interactions, but not its hydrogen-bonding properties. Likewise, a Met→Mox substitution closely resembles the electronic properties of Met. Importantly, such modified glucagon molecules have shown to reduce oxidation as compared to native glucagon and/or non-substituted phosphoglucagon derivatives of the present disclosure, thus resulting in extended shelf-life of the resulting formulations and/or pharmaceutical compositions. Furthermore, the biological activity of native glucagon is preserved in the resulting glucagon derivative.

It will be appreciated that while specific substitutions are described, any suitable oxidation resistant amino acid may be employed as long as the biological activity of the modified glucagon is significantly preserved. Especially for medical applications in use, it is desirable that the modified peptide is as close as possible to native glucagon such that it exhibits identical or substantially similar characteristics thereto. A small change may induce a significant change in physical and chemical properties of a protein, which may have a great influence in the half-life of the resulting peptide and in immunogenicity. For example, native glucagon has a half life of about 20-26 minutes for an intramuscular dose, about 30-45 minutes for a nasal powder dose, and about 28-35 minutes for a subcutaneous auto-injector or pre-filled dose. Similarly, any glucagon derivatives should be at least as (or ideally) less antigenic than native glucagon. By modifying only position 27 of the glucagon molecule, exemplary embodiments of the modified peptides hereof exhibit a half live and antigenicity that is comparable to native glucagon, while also imparting significant oxidative resistance and extending shelf-life of the resulting product. Accordingly, the modified peptide of the present disclosure is a viable substitution for native glucagon and is also capable of maintaining stability over an extended period of time which significantly enhances its shelf-life.

The modified glucagon peptide hereof may optionally comprise phosphorylation of one or more amino acids side chains involved in steric zipper formation to result in the glucagon molecule being soluble at a substantially neutral pH (as described above and in the Related Disclosures). In such embodiments, Met²⁷ of the glucagon may be substituted as described above or not; however, where the methionine is not substituted, novel antioxidant formulations may be employed to provide oxidation resistance. Such embodiments may be particularly beneficial in that they avoid potential toxicity (if any) that may result from substituting methionine Met²⁷ with methionine Nle²⁷, Mox²⁷, or any other appropriate oxidation-stable amino acid or isomer thereof. More specifically, in certain embodiments, the glucagon peptide and/or phosphoglucagon peptide (described in further detail below) may be suspended in a buffer or excipient comprising one or more antioxidants. In application, the antioxidant acts similar to a competitive inhibitor; it is present in such a concentration within the formulation that the antioxidant oxidizes first, thus protecting the methionine of the glucagon from oxidation. In other words, the antioxidants may be an oxygen scavenger that reacts with the ROS within the formulation, thereby reducing or eliminating ROS concentration within the solution.

The antioxidant utilized for the formulation may comprise any antioxidant appropriate for biological and medical formulations that is effective at a substantially neutral pH including, without limitation, ascorbic acid (e.g., L-(+)-ascorbic acid), cysteine (e.g., N-acetyl-L-cycsteine), polysorbate 20 and/or 80, ethylenediaminetetraacetic acid (EDTA), methionine (e.g., L-methionine). The concentration of the antioxidant may be adjusted as desired and according to the precise antioxidant and/or antioxidant combination employed, and may comprise, for example and without limitation, between about 0.5 mM-100 mM (inclusive of any value therein). In at least one exemplary embodiment, the concentration of antioxidant comprises about 5 mM, about 10 mM, about 15 mM or about 20 mM.

In at least one embodiment, the formulation comprises a phosphoglucagon peptide (about 1 mg/ML) prepared in PBS with about 1-5 mM EDTA. In an alternative embodiment, the formulation may comprise a phosphoglucagon peptide (about 1 mg/ML) prepared in PBS with about 0.5 mM-50 mM L-methionine, histidine buffer with about 1-5 mM EDTA, or histidine buffer with about 0.5 mM-50 mM L-methionine.

The unique peptides and formulations hereof provide several benefits of conventional approaches. Many conventional techniques employ inorganic solvents which, while perhaps acceptable for emergency rescue applications, are far from ideal for long-term, consistent metered infusions (for example, with an artificial pancreas). Further, where conventional applications do utilize organic solvents, native glucagon has been employed and the above-described issues arise with respect to solubility and stability over time. The present peptides and related formulations, compositions, and methods overcome all of the hurdles experienced with conventional approaches and provide an easy-to-use and safe alternative effective for the treatment of diabetic conditions.

The formulations comprising the novel peptides and/or buffers and excipients of the present disclosure may be for subcutaneous, intradermal, intranasal, intramuscular, or intravenous administration (e.g, by injection or by infusion). In some embodiments, the formulation is administered subcutaneously. Furthermore, the formulations of the present disclosure are administered by infusion or by injection using any suitable device. For example, a formulation of the present disclosure may be placed into a syringe, a pen injection device, a nasal spray delivery device, an auto-injector device, or a pump device. In some embodiments, the injection device is a single-dose syringe or pen device for emergency treatment of hypoglycemia. In other embodiments, the injection device is a multi-dose injector pump device or a multi-dose auto-injector device. The formulation is presented in the device in such a fashion that the formulation is readily able to flow out of the needle upon actuation of an injection device, such as an auto-injector or spray device, in order to delivery the peptide drugs. Suitable pen/autoinjector devices include, without limitation, those pen/spray/autoinjector devices manufactured by Becton-Dickenson, Swedish Healthcare Limited (SHL Group), YpsoMed Ag, and the like. Suitable pump devices include, without limitation, those pump devices manufactured by Tandem Diabetes Care, Inc., Delsys Pharmaceuticals, Medtronic MiniMed, Inc., and the like.

In some embodiments, the formulations comprising the novel peptides and/or buffers and excipients of the present disclosure are provided ready for administration in a vial, a cartridge, or a pre-filled syringe.

When the compounds of the present disclosure are administered as pharmaceuticals to humans and other mammals, they can be given per se or as a pharmaceutical composition containing, for example, about 0.1 to 99.5% (more preferably, about 0.5 to 90%) of active ingredient, i.e. at least native glucagon where the novel antioxidant formulation is employed, and/or one of methionine substituted glucagon peptides and/or other glucagon derivatives described herein, in combination with a pharmaceutically acceptable carrier. Additionally, such pharmaceuticals may also comprise antioxidant formulations where further oxidative resistance is desired.

In general, a suitable daily dose of a pharmaceutical compound of the present disclosure will be that amount of the compound that is the lowest dose effective to produce a therapeutic effect. Such an effective dose will generally depend upon the factors described above in connection with a therapeutically effective dose. If desired, the effective daily dose of the active compound may be administered as two, three, four, five, six, or more sub-doses administered separately at appropriate intervals throughout the day, optionally, in unit dosage forms.

Furthermore, the automated control of blood glucose (BG) concentration has been a long-sought goal for diabetic conditions. Conventionally, closed-loop control systems measure the BG concentration of a subject and subcutaneously deliver insulin as needed in response to the detection of increased BG levels. Due to the inability of conventional techniques to store glucagon in a biologically acceptable solution in high concentrations, a viable bi-hormonal closed-loop system capable of delivering both insulin and glucagon as needed has heretofore not been available. However, in view of the advances in glucagon solubility and stability achieved via the peptides, compositions and methods of the present disclosure, such a bi-hormonal, closed-loop system (i.e. an artificial pancreas) is now a reality. Indeed, using the novel formulations set forth herein, phosphoglucagon may be stored in high concentrations in an aqueous solution at a substantially neutral pH such that it can be automatically administered as needed by such a bi-hormonal, closed-loop system. For example, in at least one embodiment, a pharmaceutical composition may comprise a modified peptide or a pharmaceutically acceptable salt thereof.

In other aspects, the present disclosure provides kits that include stable formulations of the modified glucagon compounds hereof. For example, in at least one exemplary embodiment, the compounds of the disclosure will be stored in a vial in an aqueous solution at a substantially neutral pH (i.e. pH from 4 to and including 9). The aqueous solution will be biocompatible with humans and other mammals. In some embodiments, the kit comprises a syringe that is part of a pen injection device, an auto-injector device, a pump, or a nasal spray device. In at least one embodiment, the syringe is prefilled with the stable formulation.

Such kits may further comprise instructions. For example, such instructions may direct the administration of the stable formulation to treat the subject in need thereof (e.g., the subject experiencing acute hypoglycemia or another diabetic condition).

Methods for treating a condition using the inventive peptides and formulations hereof are also provided. In at least one embodiment, a method is provided for treating a condition or a complication thereof by administering to a subject a stable formulation comprising a modified glucagon molecule in an amount effective to treat the condition. The modified peptide may comprise any of the glucagon derivatives described herein, including a glucagon comprising a substituted methionine (e.g., switched out with an oxidative resistant methionine memetic analog). In another embodiment, the modified peptide may simply comprise a phosphoglucagon. Still further, in at least one exemplary embodiment, the modified peptide may comprise a substituted methionine and also be phosphorylated at one or more amino acids. However, it will also be noted that perhaps the glucagon may not be modified at all; instead, the benefits of the present disclosure may be achieved through a formulation comprising native glucagon suspended in an antioxidant-rich solution.

In at least one embodiment of the method, the stable formation may further comprise one or more antioxidants as described above, the inclusion of such antioxidants enhancing the stability of the modified peptide by preventing the oxidation thereof.

Furthermore, where the method is employed in connection with a bi-hormonal closed-loop system (i.e. an artificial pancreas), the method may further comprise administering insulin to the subject. In such embodiments, the stable formulation of modified glucagon and insulin are administered at different times via the system in response to the detected levels of BG in the blood. For example, where the system detects increased levels of BG as compared to an established baseline, the system will automatically administer insulin. Conversely, where the system detects decreased levels of BG as compared to an established baseline, the system will automatically administer the stable formulation of modified glucagon. It will be appreciated that the timing of such doses and the concentrations thereof can be readily determined by one of skill in the art and pursuant to defined algorithms. In this manner, the bi-directional management of a diabetic condition can be achieved. Such treatment has not been attainable to date due to the inability of conventional glucagons to be stored in a high concentration, in an aqueous solution, and/or at substantially neutral pH.

Finally, other medical applications of the modified glucagons, formulations, and kits of the present disclosure will also be realized. For example, glucagon is often used to slow or cease gastrointestinal motility in subjects who undergo gastric imaging modalities (i.e. movement of the region can result in blurred images). The benefits of the peptides and formulations discussed herein can also be useful with respect to this or any other application where it may be beneficial to employ one or more doses of glucagon that has been stored in an aqueous and substantially neutral pH for a period of time.

While various embodiments of peptides, pharmaceutical compositions, and the methods hereof have been described in considerable detail, the embodiments are merely offered by way of non-limiting examples. Many variations and modifications of the embodiments described herein will be apparent to one of ordinary skill in the art in light of the disclosure. It will therefore be understood by those skilled in the art that various changes and modifications may be made, and equivalents may be substituted for elements thereof, without departing from the scope of the disclosure. Indeed, this disclosure is not intended to be exhaustive or too limiting. The scope of the disclosure is to be defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### Phosphoglucagon Studies

In preliminary studies, singly phosphorylated glucagon analogs were custom synthesized (GenScript) using established solid-phase synthesis techniques, each phosphorylated at one of Ser², Thr⁵, Thr⁷, Ser⁸, Tyr¹⁰, Ser¹¹, Tyr¹³, and Ser¹⁶. The analogs were then assessed for solubility and stability as described below and in the Related Disclosures. While the examples below that are phosphorylated focus on phosphoglucagon derivatives containing one to two phosphate groups (which serve to demonstrate the approaches described herein), it will be appreciated that the present disclosure is not limited to phosphoglucagon derivatives containing only one or two phosphate groups, but instead also includes higher levels of derivatization.

### Example 1

### Solubility of Phosphoglucagons

An ideal glucagon for hypoglycemic rescue would have adequate solubility in aqueous solution at a neutral pH. The approximate solubilities of native human glucagon and its phosphoglucagon analogs were measured at room temperature by the drop-wise addition of 50 mM phosphate buffer (pH 7.4) or 50 mM phosphate-buffered saline (pH 7.4) to a known amount of peptide until complete dissolution resulted (as confirmed by visual observation).

More specifically, for turbidity measurements, 100 μL of filtered stability samples were transferred to a 96-well microtiter plate (in triplicate), final volume was made up to 200 μL with 50 mM sodium phosphate (pH 7.4), and UV absorbance at 405 nm and 280 nm were used to calculate an aggregation index. Turbidity is reported as the time in days required to increase turbidity by 50% of the initial value. For fluorescence measurements, glucagon and phosphoglucagon solutions were prepared at 1 mg/mL in either 3.2 mM HCL, 0.9% NaCl (w/v) (pH 2.5) or 50 mM sodium phosphate (pH 7.4), samples were centrifuged and filtered, placed in a 96-well black flat bottom microtiter plate in triplicate, and incubated with 50 μM Thioflavin-T (ThT) final concentration. To determine the fluorescence intensity of ThT, the excitation and emission wavelengths were set at 440 nm and 482 nm, respectively. To determine changes in fluorescence emission from Trp-25 (intrinsic fluorescence, IF), the excitation and emission wavelengths were set to 295 nm and 355 nm, respectively. All fluorescence readings were carried out at 15 min intervals for 24 hrs and fluorescence signals of over 100,000 (overflow) were set to 100,000 for visualization purposes. Results for ThT and IF are reported as time needed to reduce signal by 50% compared to initial reading.

The volume of buffer required to completely dissolve a known amount of peptide was used to calculate the peptide concentration in mg/mL (Table 1). The standard dose of glucagon for rescue is 1 mg and is delivered in 1 mL of solution; therefore, 1 mg/mL served as the target solubility for these studies.

TABLE 1
Summary of solubility and stability measurements on phosphoglucagon analogs.

| | | | ThT | IF | Turbidity |
|---|---|---|---|---|---|
| | Approx. Solubility (mg/ml) ^{a} | | T₅₀ (hrs.) | T₅₀ (hrs.) | T₅₀ |
| Glucagon analogs | PB pH 7.4 | PBS pH 7.4 | pH 7.4^{b} | pH 7.4^{b} | pH 7.4^{b} |
| native Glucagon | <0.1 | <0.1 | NS | NS | |
| phospho-Ser²-glucagon | 0.8 | <0.5 | — | — | |
| phospho-Thr⁵-glucagon | 5.6 | 2.2 | ND | ND | ND |
| phospho-Thr⁷-glucagon | 4.2 | 1.6 | >24 | >24 | |
| phospho-Ser⁸-glucagon | 6 | 2.1 | ND | ND | ND |
| phospho-Tyr¹³-glucagon | 1.5 | 0.9 | >24 | >24 | |
| phospho-Tyr¹⁰-glucagon | <0.5 | <0.1 | NS | NS | |
| phospho-Ser¹¹-glucagon | <0.1 | <0.1 | NS | NS | |
| phospho-Ser¹⁶-glucagon | 3.7 | 1.6 | — | — | |

^{a} Solubility in 50 mM sodium phosphate (PB), pH 7.4 at room temperature.
^{b}Performed in 50 mM, pH 7.4.
PB = phosphate buffer.
NS = not soluble.
ND = not detected during 35-day study period.

The solubility studies demonstrated that many phosphoglucagon analogs exhibited high solubility (>1 mg/mL) at neutral pH while, as expected, native glucagon was essentially insoluble at this pH.

### Example 2

### Stability of Phosphoglucagons

In addition to solubility at a neutral pH, the medical impact and commercial viability of phosphoglucagon depend on its stability in solution. Phospho-Thr⁵-, phospho-Thr⁷-, and phospho-Ser8-glucagon were selected for stability studies, involving assessments of physical stability, structural stability, and chemical stability, as they had the greatest solubility of the phosphoglucagon analogs. For stability studies, phosphoglucagon solution samples were prepared at 1 mg/mL in 50 mM sodium phosphate (pH 7.4), centrifuged at 14,000 rpm for 5 min, and filtered through 0.1 μm filters to remove any insoluble material. The samples were aliquoted as 300 μl into 2 mL vials, sealed under nitrogen gas and stored in a dark place at room temperature for 35 days. Vials were withdrawn at regular intervals to monitor physical stability using turbidity measurements; structural stability by far-UV circular dichroism (CD) spectroscopy and fluorescence measurements; and chemical stability by liquid chromatography mass spectrometry (LC/MS).

More specifically, stability samples were diluted in 0.1% formic acid (FA) and approximately 60 pmole of phosphoglucagon was injected into a peptide microtrap. Samples were desalted for 2 min with 15% acetonitrile, 85% water, and 0.1% FA. Mass spectra were obtained over the m/z range 100-1700, using a ESI-LC/MS system (1200 series LC, 6520 Q-TOF). The raw data were processed, and the mass analyzed using the data analysis software (MassHunter Software). Met²⁷ oxidation was quantified by measuring the peak height of oxidized species relative to the non-oxidized species in the mass spectra.

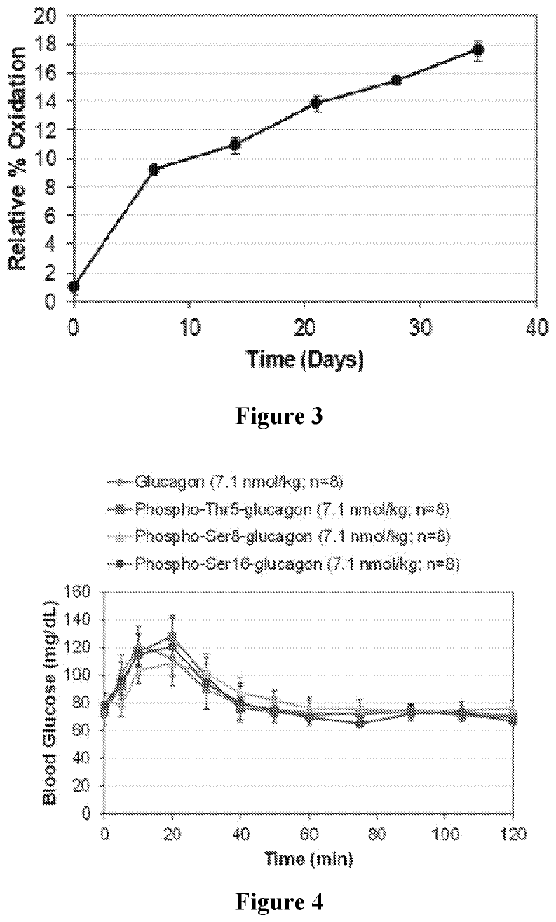

As shown in Table 1, all three phosphoglucagon analogs displayed excellent stability with phospho-Thr⁵- and phospho-Ser8-glucagon having no detectable fibrillation during the 35-day testing period. CD spectroscopy showed that the structure of the phosphoglucagons is virtually unchanged from day 1 to day 35 of the stability study. While the physical and structural stability of the phosphoglucagons were favorable, LC/MS analysis revealed a detectable level of methionine (Met²⁷) oxidation (see FIG. 3). Embodiments of the present disclosure described below address this impurity using inventive formulation strategies to minimize oxidation reactions and/or through modifications of the phosphoglucagon sequence to replace the methionine residue.

### Example 3

### Dephosphorylation of Phosphoglucagons

To evaluate whether glucagon phosphorylation can be reversed by exposure to phosphatase enzymes, the kinetics of de-phosphorylation was examined using phospho-Thr⁵- and phospho-Ser⁸-glucagon. For this study, a colorimetric phosphatase assay (BIOMOL) was carried out, in which free phosphate reacts with the BIOMOL green reagent to produce a color change (yellow to green) that is directly proportional to the free phosphate concentration. Specifically, 2 nmol of analogues were separately incubated with 0.009 units of bovine alkaline phosphatase in assay buffer (50 mM Tris, pH 7.4) to a final volume of 50 μL. The reaction was carried out in a 96-well crystal-clear microtiter plate over 5-480 min at 37° C. The reaction was quenched by adding 100 μL of BIOMOL green reagent (malachite green) and read at 620 nm. Samples with known phosphate concentrations were used to obtain a phosphate standard curve.

TABLE 2
Summary of dephosphorylation study.

| | Dephosphorylation |
|---|---|
| | T₅₀ (mins.) |
| Glucagon analogs | pH 7.4 |
| phospho-Thr⁵-glucagon | 85.9 |
| phospho-Ser⁸-glucagon | 94.9 |

The results support that de-phosphorylation occurs readily. Indeed, as shown in Table 2, within approximately 1.5 h, roughly half of the phosphoglucagon had been dephosphorylated.

### Example 4

### In Vivo Activity of Phosphoglucagons

To demonstrate that phosphorylation does not inhibit or prevent the pharmacological activity of glucagon, the ability of representative phosphoglucagons to increase blood glucose levels in rats was compared to the elevation caused by native glucagon.

The phosphoglucagon analogs were dialyzed in 50 mM sodium phosphate buffer (pH 7.4). Thereafter, approximately 7.1 nmol/kg of either native glucagon or phosphoglucagon was subcutaneously injected into male Wistar rats that had been fasted for 16 hrs. The total blood glucose level was measured by withdrawing blood at regular intervals (5-120 min) and tested using Freestyle Lite® glucose test meters (Abbott).

FIG. 4 shows the blood glucose measurements taken in the rats in response to the administration of native glucagon or phosphoglucagon. Importantly, the phosphoglucagons increased fasted blood glucose to similar levels as compared to native glucagon and that the increase occurred at comparable rates. As such, this data supports the inventive phosphoglucagons of the present disclosure exhibit comparable performance in vivo to native glucagon.

Accordingly, the phosphoglucagon data presented above an in the Related Disclosures demonstrate the feasibility of using phosphoglucagon in a stable, solution formulation for hypoglycemic rescue methodologies and related kits. Specifically, such data establishes several phosphorylation sites on glucagon that: (1) provide adequate solubility at neutral pH (>1 mg/mL), (2) inhibit fibrillation in vitro, and (3) effect blood glucose elevation in rats that is comparable to that effected by native glucagon.

### Oxidative Stability Studies

With the foregoing phosphoglucagon data as a backdrop, stability issues due to methionine oxidation were then addressed with the goal of expanding the shelf-life of both glucagon and/or phosphoglucagon formulations. Four phosphoglucagon candidates identified in preliminary efforts were modified to improve their oxidative stability using two separate approaches: (1) modifying the phosphoglucagon sequence to replace Met²⁷ with norleucine (Nle²⁷); and (2) evaluating formulation strategies to minimize oxidation in unmodified phosphoglucagons. The resulting formulations and Nle²⁷-modified phosphoglucagons were then evaluated for in vitro stability and in vivo functionality, with the phosphoglucagons ranked according to their adherence to predetermined metrics for stability and functionality.

### Example 5

### Optimizing Phosphoglucagon and Native Glucagon Stability Through Formulation and Structural Modifications

Despite phosphorylation, the phosphoglucagon analogs identified herein have shown oxidation of Met²⁷ to methionine sulfoxide and (to a lesser extent) methionine sulfone after 30 days of storage in unprotected formulations. To extend the shelf-life of the formulation and make phosphoglucagons and/or native glucagon amendable to other applications (such as for use in an artificial pancreas), the stability of the current formulation must be extended. The desired enhanced stability was achieved through changes in the formulation (i.e. the addition of particular buffers and/or excipients) and/or modifications of the methionine residue of the glucagon amino acid chain.

To demonstrate enhanced stability is achieved through the substitution of Met²⁷ with norleucine, the three lead phosphoglucagons (phospho-Thr⁵-glucagon, phospho-Thr⁷-glucagon, and phospho-Ser⁸-glucagon) were synthesized both with and without a norleucine substitution for the methionine (i.e. Met²⁷→Nle²⁷). All peptides were custom synthesized by GenScript using established solid-phase synthesis techniques and thereafter formulated in two buffers—Histidine and 1×PBS—to a concentration of 1 mg/mL. The solubility of the Nle-analogs was estimated, and any fibrillation monitored using previously described techniques, using native glucagon as the control.

In parallel to the Met²⁷ to Nle²⁷ modification study, several buffers and excipients were evaluated for their ability to inhibit oxidation of Met²⁷ using a phosphoglucagon known to oxidize (e.g., phospho-Ser⁸-glucagon, see FIG. 3). Specifically, a sample of each of the three phosphoglucagons identified above were prepared in 1 mg/mL solutions in the following buffer: PBS with 1-5 mM EDTA. Solubility and fibrillation (30 day at room temperature in the dark) were assessed as described in the phosphoglucagon studies set forth above. Buffers that successfully inhibited oxidation of phospho-Ser⁸-glucagon were then evaluated in connection with the other lead phosphoglucagons.

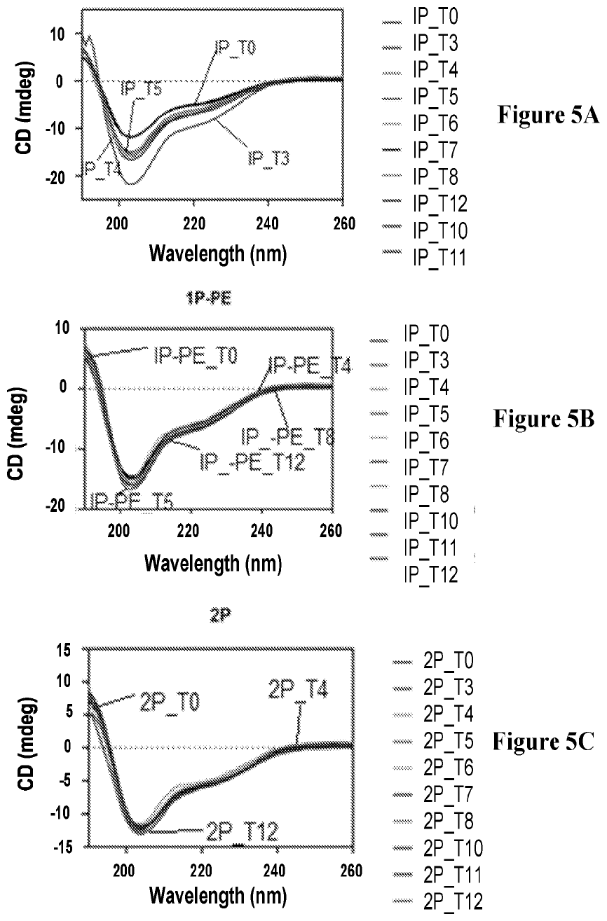

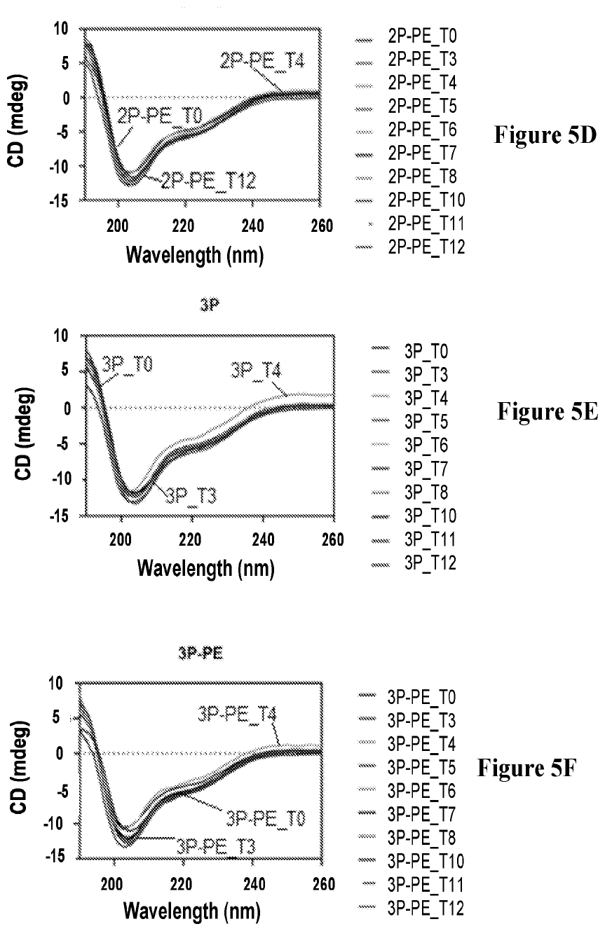

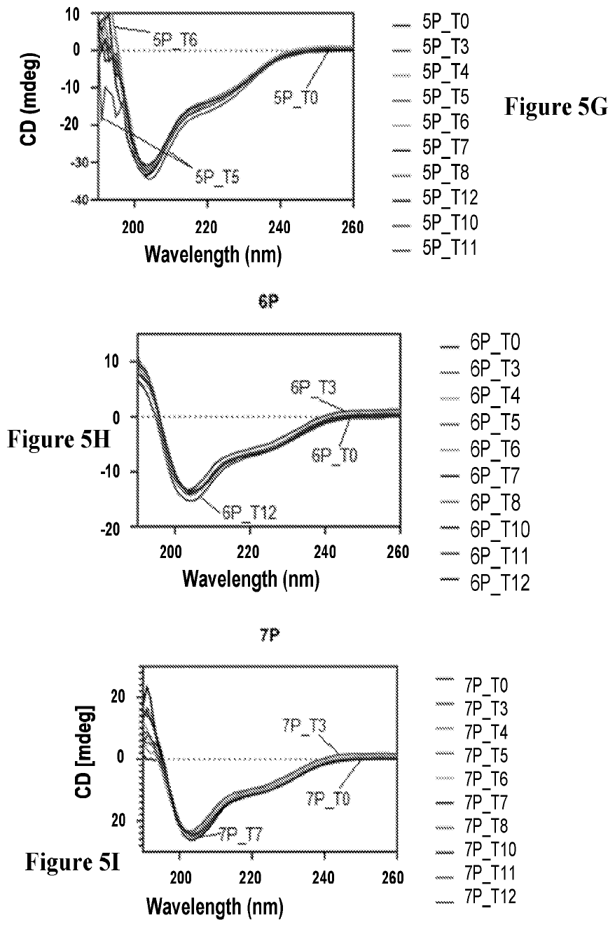

FIGS. 5A-5I illustrate the results of such studies. In both the Met²⁷ substitution and buffer/excipient studies (FIGS. 5B, 5D, and 5F-5I), the phosphoglucagon derivatives were able to achieve a desirable and improved stability at neutral pH as compared to previous iterations and conventional techniques. Indeed, less than 10% oxidation was observed in the samples after 3 months at 30° C., with either no or negligible fibrillation detected. Furthermore, solubility of greater than or equal to about 1 g/mL at a neutral pH was achieved in the test samples.

### Example 6

### Stability and In Vivo Activity of Methionine Substituted Glucagon Analogs

The native glucagon and the top formulations from Example 5 that exhibited greater than or equal to about 1 mg/mL of solubility at neutral pH and had little to no oxidation or fibrillation over 30 days were assessed for 3-month stability and in vivo activity.

The methionine substituted peptides indicated above were synthesized by GenScript, with each comprising of a modified glucagon and/or phosphoglucagon derivatives with methionine substituted for norleucine. Specifically, the one glucagon and eight phosphoglucagon formulations identified above were prepared, aliquoted into vials, and placed at 30° C. for 3 months to assess stability. Three vials of each formulation were pulled weekly for stability analysis, with the extent of fibrillation monitored using intrinsic fluorescence, ThT fluorescence and turbidity (UV) measurements taken (as in the preliminary studies), and all compared to a glucagon control. Fibrillation was also monitored using size exclusion chromatography (SEC) as loss of the parent peak.

For detection of oxidation products, ESI LC/MS was used to identify methionine sulfoxide (+16) and/or methionine sulfone (+32). The extent of oxidation was quantified as the relative area of oxidation products on EIC as a fraction of the total area of peptide species. Differences in stability between time points and formulation strategies was then determined using ANOVA followed by a test for multiple comparisons (Duncan's).

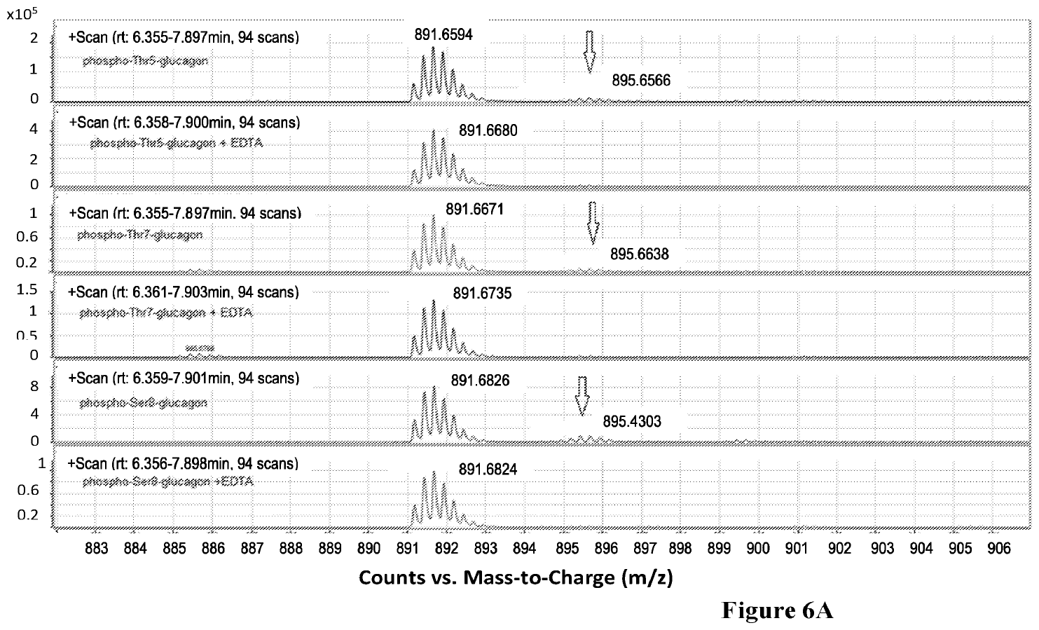

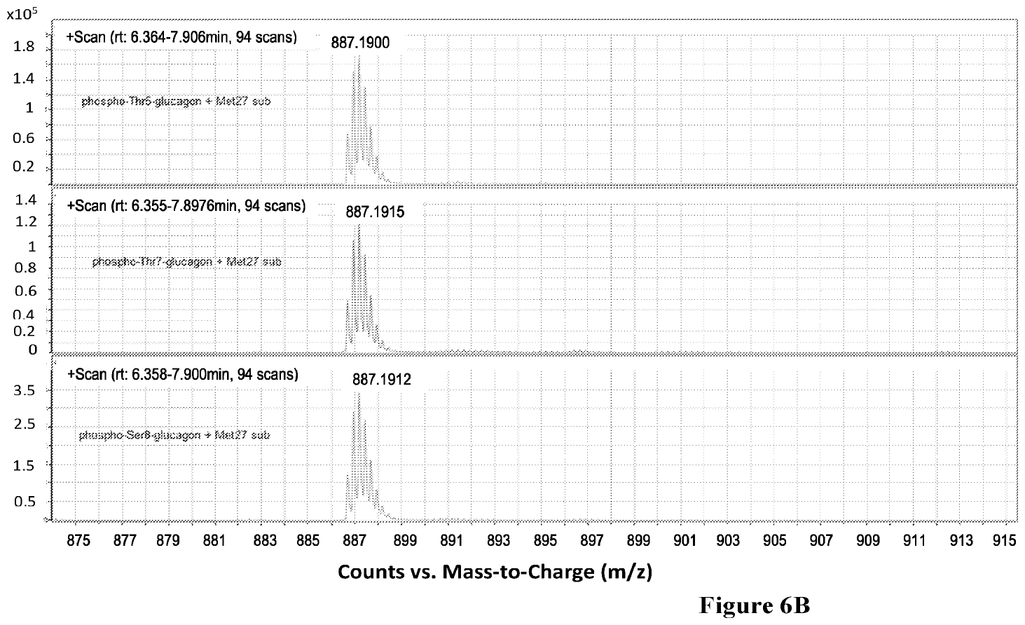

Of the samples tested, only the three (3) phosphoglucagons that were not methionine substituted or in the antioxidant solution displayed significant oxidation (indicated by arrows in FIG. 6A). The remaining methionine substituted samples and those that were not methionine substituted but within an antioxidant solution displayed less than about 10% oxidation when stored at 30° C. for three months, and showed no or negligible fibrillation during this time period. (see FIGS. 6A and 6B).

Stability assessments were then performed on the various formulations (inclusive of both the formulation changes and norleucine substitution formulations). In addition to the measurable differences observed as compared to native glucagons with respect to one approach or the other, it will also be noted that the combination of these approaches achieved significantly enhanced stability in the resultant formulations.

### Example 7

### Determine the Pharmacodynamic Properties and Functionality of Modified Phosphoglucagons Using Rats

Phosphoglucagon formulations from Example 6 (e.g., phospho-Thr⁵-glucagon+EDTA; phospho-Thr⁵-glucagon+Met²⁷ substitution; phospho-Thr⁷-glucagon+EDTA; phospho-Thr⁷-glucagon+Met²⁷ substitution; phospho-Ser⁸-glucagon+EDTA; phospho-Ser⁸-glucagon+Met²⁷ substitution) were then assessed in vivo to verify the peptide exhibits full in vivo biological activity (i.e. that it increases blood sugar at a rate similar to native glucagon, with a rapid onset of action and short duration of action). This was assessed with rats using methods described in the preliminary results section with respect to the phosphoglucagon studies. Eight Wistar rats (4 male and 4 female; fasted 16 hours) were used per phosphoglucagon formulation, native glucagon, and vehicle control and all treatment was delivered through intramuscular injection. Time-to-peak blood glucose level (tₘₐₓ), peak blood glucose level (Cₘₐₓ) and duration of action (e.g., time to return to about +/−10% of baseline blood glucose) was determined for each formulation and compared as described in the previously described studies.

The phosphoglucagon samples comprising modified methionine increased blood glucose similar to native glucagon (rate and extent) were identified, as did (unsurprisingly) the phosphoglucagons without methionine substitutions (see previous phosphoglucagon studies). Specifically, the quantitative benchmarks for in vivo response following ˜7 nmol/kg IM dose in rats were: (i) blood glucose elevation of at least 40 mg/dL in ≤ about 15 min and (ii) return to +/−10% of baseline blood glucose level in ≤ about 2 h.

### Example 8

### The Development and Validation of Analytical Methods to Detect and Quantify Phosphoglucagon in Plasma

To demonstrate the reliability of the phosphoglucagon detection methods and ultimately support PK studies, methods for detecting and quantifying phosphoglucagon with modified methionine in plasma were developed, with reproducibility and bias to be <20% CV for all metrics. Such method development focused on an LC-MS approach that was subsequently validated using plasma collected from rats that has been administered the phosphoglucagons described herein.

A multiplexed LC-MS/MS assay was used to measure the phosphoglucagon prodrug candidates, as well as the corresponding dephosphorylated glucagon and glucagon analogs, following published methods. The peptide analytes were isolated from plasma by protein precipitation using organic solvents and solid phase extraction (SPE) using ion exchange stationary phases at predetermined optimal protein precipitation and solid phase extraction conditions. The precise strategy for isolating glucagon was determined through screening a variety of extraction solvents and solid phase extraction conditions. Further, because glucagon is likely to exist in a number of charge states, the optimal charge state for use in the measurements of each analog was assessed and ranked based on signal intensity and stability.

Analyte recovery was optimized through solvent screening and any issues with assay performance were readily corrected by means of an internal standard. A direct analysis of the analytes was performed using high-resolution mass spectrometry, with the assay designed to have a dynamic range of 5000-50 ng/mL in plasma, which is sufficient for the determination both the Cₘₐₓ and steady state levels. Ideal embodiments of this LC-MS method had a limit of detection of 50 nm/mL for phosphoglucagon in a plasma matrix, which allows for a range that encompasses five half-lives of the peptide.

To validate this analytical approach, four phosphoglucagon candidates were selected, along with one non-phosphorylated glucagon control, for assessment. All samples were spiked into rat plasma at concentrations ranging from 50 ng/mL to 5000 ng/mL, extracted via solid phase extraction, then analyzed by LC-MS. The assay was evaluated for reproducibility, peptide stability, linearity, lower limit of quantification, and interferences. Reproducibility was determined by means of multiple injections over multiple days, with interday, intraday, and total CVs determined.

For peptide stability, bias and CV of triplicate samples were compared to extrapolated values, with the lower limit of quantitation set at no less than 3× the noise. Interference was determined by the addition of clinically relevant potential interferents. The CV of triplicate spiked samples and bias when accounting for dilution of spiking (5%-50% dilution depending on interferent solution) was also compared to expected values. Any issues associated with sample stability were addressed by the use of protease inhibitors and reduced temperature sample handling techniques.

### Example 9

### Long-Term Stability Studies of Phosphoglucagon Formulations

The following 4 modified phosphoglucagons comprising modified methionine from Example 8 were then prepared and aliquoted for long-term stability studies. Samples of each derivative were stored at 4° C., 25° C., and 40° C. for 18 months with six replicates of each sample being removed from storage monthly and assessed for stability pursuant to the protocol described in Example 6 above. Differences in stability measurements between time points and formulations strategies were assessed using ANOVA, followed by a test for multiple comparisons (Duncan's).

The metric for success for this study was to identify formulations that display less than 10% oxidation when stored at 40° C. and less than 2% when stored at 4° C., both for a period of 18 months. Additionally, no fibrillation should be detected during this time period. Alternative storage vials and modified storage conditions were also considered with respect to potentially effecting stability of the inventive formulations.

### Example 10

### Assessment of PK/PD Properties of Phosphoglucagon Formulations

To assess the kinetics of the lead four phosphoglucagons comprising modified methionine in rats, the PK/PD properties of each of the four modified phosphoglucagons were evaluated in vivo via both intranasal (IN) or intramuscular (IM) delivery (2 for IN and 2 for IM). The two most stable and soluble candidates were evaluated as IN agents and the other two candidates were assessed for IM administration. While IM delivery of native glucagon is well-characterized and can serve as a benchmark for the IM candidates, IN is less characterized; therefore, additional doses for the IN studies were evaluated to ensure the PD properties were fully defined.

Prior to administration, fasted (16 hrs) Wistar rats were catheterized via jugular catheters to enable collection of blood samples at various time points post-dosing. Each rat received a single dose of each modified phosphoglucagon through the appropriate route (IM or IN; 4 males and 4 females/group). Groups receiving vehicle or native glucagon (7.1 nmol/kg) served as controls. For the modified phosphoglucagons delivered via IM, about 2.5, 5.0, 7.1, or 10 nmol/kg of modified phosphoglucagon was intramuscularly injected into conscious rats. Similarly, six concentrations of IN were evaluated, with about 5.0, 7.1, 10, 15, 20, and 40 nmol/kg of modified phosphoglucagon delivered via a pipette into the left nostrils of rats in the IN group under anesthesia (3% isoflurane at 3 L/min O₂ flow rate) to ensure delivery of the entire dose. A larger dose range was assessed in the IN group to allow for the smaller volume of drug being delivered. Following IN dosing, the animals were held in the vertical position for a minimum of 30 sec to allow the dosing solution to flow through the sinus cavities.

The top phosphoglucagon formulation for each delivery route (IM and IN) that satisfied the defined study metrics, were flagged and advanced to the preliminary safety and immunogenicity profile studies described below. The total blood glucose level was measured by withdrawing blood at regular intervals (5-120 min) and tested using FREESTYLE LITE (glucose test meters, Abbott, Chicago, Ill.). Blood samples were collected prior to dosage and at 10 time points post-dosing (baseline, every 10 min until 80 min, then at 120 min).

Certain samples had similar profiles to native glucagon in regards to 1) the extent of the elevation of blood glucose; 2) the time required to reach the peak blood glucose level; and 3) the time to reach baseline (trough) levels. In vivo, the concentrations of the modified phosphoglucagons and dephosphorylated prodrug in the plasma samples from the group receiving the lowest efficacious dose (as determined by the blood glucose measurements) was flagged for further studies, including an assay. ANOVA with Duncan's was also employed to determine the significances of differences in time points relative to the baseline or fasting glucose value.

### Example 11

### Toxicity Studies

The top modified phosphoglucagons identified in Example 10 for each route were then assessed to evaluate toxicity at the site of administration. Primarily, to determine the local inflammatory response of each selected modified phosphoglucagon following IN and IM administration, for the IM treatments, about 2× the effective concentration of that used in Example 10 of each of the advanced modified phosphoglucagons was subcutaneously (SC) injected into male and female Sprague-Dawley rats (6 injections/animal; 3 males and 3 females) under anesthesia (inhaled 3% isoflurane at 3 L/min O₂ flow rate). Specifically, each rat had a grid (2 squares wide by 3 squares high) drawn on its shaved back and a single SC injection was administered into each grid square. Further, about 10× the effective concentration of the that used in Example 10 of each of the advanced modified phosphoglucagons was delivered into the left nostril of 2 male and 2 female Sprague-Dawley rats under anesthesia (inhaled 3% isoflurane at 3 L/min O₂ flow rate). Following IN dosing, the animals were held in the vertical position for a minimum of 30 sec to allow the dosing solution to flow through the sinus cavities. Groups receiving vehicle only served as controls.

Four hours after dosing, animals that received IN treatments were anesthetized and underwent nasal lavage with 1×PBS. The lavage was centrifuged and the pellet resuspended and applied to a cytospin column to concentrate the sample for cell count and differential analyses. Additionally, nasal turbinates were collected for histology.

The animals that received SC treatments were euthanized, their back skin removed and cleaned of fat and fascia to allow for the assessment of any irritation present thereon. A subjective score ranging from 0 to 3 based on the level of redness and inflammation observed was generated, with 0 being no reaction and 3 being the greatest reaction. Skin samples were also processed for histology. Histology and subjective scoring was blind.

The candidates did not result in severe tissue site inflammatory reactions (i.e. scores less than 3). IN administration is more likely to result in an undesirable reaction as compared to IM delivery; however, the candidates that did not induce severe tissue site inflammatory reactions likely do not because 1) it was rapidly dephosphorylated to native glucagon in vivo; 2) the modified phosphoglucagon has minor modifications resulting in safety profiles similar to native glucagon; and 3) the formulation is not designed for slow release or multiple dosing.

### Example 12

### Anti-Drug Antibodies and Immunogenicity Studies

The top modified phosphoglucagons identified in Example 10 for each route were also assessed with respect to immunogenicity potential; namely, to assess the production of antibodies that could prevent drug activity. Plasma samples were retained from the histological determination of acute toxicity in Example 11 for future analysis. Where histological signs of inflammation were identified, protein A/G was used to enrich immunoglobulins from the corresponding plasma samples. Once enriched, a glucagon detecting antibody was added, resulting in a sandwich ELISA. Positive signals suggest the presence of anti-drug antibodies and neutralizing antibodies to native glucagon resulting from the administration of the modified phosphoglucagon. Anti-glucagon antibodies at known concentrations added prior to the detecting antibody served as a positive control and assay validation, with a signal three times the noise level used to determine the lower limit of quantification.

1. A peptide comprising the amino acid sequence of SEQ ID NO: 1 modified such that the amino acid at position 27 is substituted with an oxidation resistant methionine memetic analog or an isomer thereof.

2. The peptide of claim 1, wherein the methionine memetic analog comprises a norleucine or an isomer thereof, or methoxinine or an isomer thereof.

3. The peptide of claim 2, comprising SEQ ID NO: 2, wherein X comprises norleucine or an isomer thereof, or methoxinine or an isomer thereof.

4. The peptide of claim 1, further comprising one or more phosphorylated amino acids.

5. The peptide of claim 4, wherein the one or more phosphorylated amino acids are selected from the group consisting of His¹, Ser², Thr⁵, Thr⁷, Ser⁸, Tyr¹⁰, Ser¹¹, Tyr¹³, Ser¹⁶, Thr²⁹, and combinations thereof.

6. A pharmaceutical composition comprising:
a modified peptide or a pharmaceutically acceptable salt thereof, the modified peptide comprising the amino acid sequence of SEQ ID NO: 1 modified such that (a) the amino acid at position 27 is substituted with an oxidation resistant methionine memetic analog or an isomer thereof, (b) one or more of the amino acids of the modified peptide are phosphorylated, or (c) both (a) and (b); and
a pharmaceutically acceptable carrier.

7. The pharmaceutical composition of claim 6, wherein the modified peptide comprises one or more phosphorylated amino acids and the pharmaceutical composition further comprises an antioxidant.

8. The pharmaceutical composition of claim 6, wherein the one or more phosphorylated amino acids are selected from the group consisting of His¹, Ser², Thr⁵, Thr⁷, Ser⁸, Tyr¹⁰, Ser¹¹, Tyr¹³, Ser¹⁶, Thr²⁹, and combinations thereof.

9. The pharmaceutical composition of claim 7, wherein the composition is a prodrug.

10. The pharmaceutical composition of claim 9, wherein each phosphate group is chemically or enzymatically cleaved upon administration of the prodrug.

11. The pharmaceutical composition of claim 7, wherein the antioxidant is selected from a group consisting of: ascorbic acid, cysteine, polysorbate 20, polysorbate 80, ethylenediaminetetraacetic acid (EDTA), methionine, and an isomer of any of the foregoing antioxidants.

12. The pharmaceutical composition of claim 11, wherein the pharmaceutical composition comprises phosphate-buffered saline (PBS) with 1-5 mM EDTA suspended therein, PBS with 0.5 mM-50 mM L-methionine suspended therein, histidine buffer with 1-5 mM EDTA suspended therein, or histidine buffer with 0.5 mM-50 mM L-methionine suspended therein.

13. The pharmaceutical composition of claim 6 comprising an aqueous solution at a substantially neutral pH.

14. The pharmaceutical composition of claim 6 comprising the modified peptide in a concentration of at or between 1 mg/mL-50 mg/mL.

15. A method of treating a condition, the method comprising:
treating a condition or a complication thereof by administering to a subject a stable formulation comprising a modified peptide or a pharmaceutically acceptable salt thereof in an amount effective to treat the condition and a pharmaceutically acceptable carrier;
wherein the modified peptide or pharmaceutically acceptable salt thereof is comprising the amino acid sequence of SEQ ID NO: 1 modified such that (a) an amino acid at position 27 is substituted with an oxidation resistant methionine memetic analog or an isomer thereof, (b) one or more amino acids of the glucagon are phosphorylated, (c) or both (a) and (b).

16. The method of claim 15, wherein the modified peptide or pharmaceutically acceptable salt thereof comprises SEQ ID NO: 2, wherein X is norleucine or an isomer thereof or methoxinine or an isomer thereof.

17. The method of claim 15, wherein the stable formulation further comprises one or more antioxidants selected from the group consisting of: ascorbic acid, cysteine, polysorbate 20, polysorbate 80, ethylenediaminetetraacetic acid (EDTA), methionine, or an isomer of any of the foregoing.

18-19. (canceled)

20. The method of claim 15, further comprising administering insulin to the subject.

21. The method of claim 20, wherein the stable formulation of modified glucagon and insulin are administered at different times via a device that monitors blood glucose levels of the subject and doses the two drugs independently as needed.

22. The method of claim 15, wherein the condition comprises a diabetic condition or gastrointestinal motility.

23-26. (canceled)


SEQUENCE LISTING (2 sequences)

SEQ ID NO: 1 - protein, 29 aa, Homo sapiens
HSQGTFTSDYSKYLDSRRAQDFVQWLMNT

SEQ ID NO: 2 - protein, 29 aa, Artificial Sequence
  Methionine substituted glucagon, where X is a memetic analog of methionine or an isomer thereof
  misc_feature (27)..(27): Xaa can be any naturally occurring amino acid
HSQGTFTSDYSKYLDSRRAQDFVQWLXNT